(19) United States
(12) Patent Application Publication
(10) Pub. No.: US 2025/0000423 A1
(43) Pub. Date: Jan. 2, 2025

(54) DEVICE FOR DIAGNOSING ETIOLOGY, STORAGE MEDIA AND ELECTRONIC DEVICE

(71) Applicant: FUWAI HOSPITAL CHINESE ACADEMY OF MEDICAL SCIENCES, Beijing (CN)

(72) Inventors: Yan YAO, Beijing (CN); Jiao LI, Beijing (CN); Si ZHENG, Beijing (CN); Xiaowei XU, Beijing (CN); Zhao HU, Beijing (CN)

(73) Assignee: FUWAI HOSPITAL CHINESE ACADEMY OF MEDICAL SCIENCES, Beijing (CN)

(21) Appl. No.: 18/694,878

(22) PCT Filed: Jun. 30, 2023
(86) PCT No.: PCT/CN2023/104671

§ 371 Date: Mar. 22, 2024

(30) Foreign Application Priority Data
Feb. 2, 2023 (CN) 202310051614.1

(51) Int. Cl. A61B 5/363; A61B 5/00

(52) U.S. Cl. CPC A61B 5/363; A61B 5/7264

(57) ABSTRACT

A device for diagnosing etiology, a storage media and an electronic device are provided according to the present disclosure. The device for diagnosing etiology comprises: a first determination unit, configured to determine electrocardiogram information corresponding to a patient; a judgment unit, configured to determine whether the patient meets a preset condition; an information processing unit, configured to access case data of the patient in case that the patient meets the condition, input the case data into an etiology diagnosis information model, and obtain etiology diagnosis information after processing; a second determination unit, configured to determine at least one target etiology diagnosis model from a first etiology diagnosis model and a second etiology diagnosis model; an etiology prediction unit, configured to input the etiology diagnosis information into the target etiology diagnosis model, and obtain diagnosis information after processing; and a third determination unit, configured to determine an etiology diagnosis result based on the diagnosis information. The device of the present disclosure can be applied to assist doctors in diagnosing etiology of sustained monomorphic ventricular tachycardia through an etiology diagnosis model, which improves the diagnosis accuracy.

This application claims the priority to Chinese Patent Application No. 202310051614.1, titled “DEVICE FOR DIAGNOSING ETIOLOGY, STORAGE MEDIA AND ELECTRONIC DEVICE”, filed on Feb. 2, 2023 with the China National Intellectual Property Administration, which is incorporated herein by reference in its entirety.

### FIELD

The present disclosure relates to the field of digital medical technology, and in particular to a device for diagnosing etiology, a storage media and an electronic device.

### BACKGROUND

With the development of computer technology, various digital auxiliary diagnostic technologies have been widely used in the clinical diagnosis field. Clinical decision support system (CDSS) is one of the key focuses of attention and development for various medical institutions. CDSS can be used to assist clinicians in disease diagnosis.

Sustained monomorphic ventricular tachycardia (SMVT) is a common arrhythmia disease in clinical diagnostic scenario. Currently, there is still a lack of a CDSS that can assist clinicians in conducting etiological diagnosis of SMVT. The etiological diagnosis of SMVT still relies on the personal determination of clinicians.

Based on etiological diagnosis methods of SMVT in conventional technology, the diagnosis result depends on the clinical experience and mastery of medical knowledge of the clinicians. In practical clinical diagnostic scenario, those undertaking frontline work are not only experienced doctors with solid medical knowledge but also residents or less experienced junior doctors. In the diagnosis process, due to differences in experience of the clinicians and the heavy workload, it is more likely to overlook key information, resulting in lower accuracy of the diagnosis result.

### SUMMARY

In view of this, according to the embodiments of the present disclosure, a method for diagnosing etiology is provided, which addresses the existing problem that it is more likely to overlook key information and compromise diagnosis accuracy when relying on manual experience for diagnosing etiology of SMVT.

A device for diagnosing etiology is further provided according to the embodiments of the present disclosure, to ensure the practical implementation and application of the above method.

Provided is a method for diagnosing etiology, comprising:
determining electrocardiogram information corresponding to a patient in case that an etiology diagnosis for the patient is required;
determining, based on the electrocardiogram information, whether the patient meets a preset diagnosis condition for sustained monomorphic ventricular tachycardia;
accessing, in case that the patient meets the diagnosis condition for sustained monomorphic ventricular tachycardia, case data of the patient, and inputting the case data into an established etiology diagnosis information model; and obtaining, through the etiology diagnosis information model, etiology diagnosis information corresponding to the patient;
determining a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method;
inputting, for each target etiology diagnosis model, the etiology diagnosis information into the target etiology diagnosis model; and obtaining, through the target etiology diagnosis model, diagnosis information outputted by the target etiology diagnosis model, wherein the diagnosis information comprises a plurality of predicted etiologies and predicted probabilities of respective predicted etiologies; and
determining, based on the diagnosis information outputted by each target etiology diagnosis model, an etiology diagnosis result corresponding to the patient.

According to the above method, optionally, the determining, based on the electrocardiogram information, whether the patient meets a preset diagnosis condition for sustained monomorphic ventricular tachycardia, comprises:
determining a judgment mode selected by a user;
in case that the judgment mode is a preset manual judgment mode, determining whether the patient meets a preset symptom condition based on the electrocardiogram information;
in case that the patient meets the preset symptom condition, generating prompt information and accessing preset identification knowledge information, wherein the prompt information is configured to prompt the patient has a symptom of sustained monomorphic ventricular tachycardia;
feeding back the prompt information, the identification knowledge information and the electrocardiogram information to the user, and prompting the user to input a judgment result whether the patient has sustained monomorphic ventricular tachycardia; and
in case that the judgment result inputted by the user indicates that the patient has sustained monomorphic ventricular tachycardia, determining that the patient meets the diagnosis condition for sustained monomorphic ventricular tachycardia.

According to the above method, optionally, the method further comprises:
in case that the judgment mode is a preset intelligent judgment mode, accessing an electrocardiogram contained in the electrocardiogram information;
inputting the electrocardiogram into an established electrocardiogram diagnosis model, and obtaining an identification result outputted by the electrocardiogram diagnosis model upon processing by the electrocardiogram diagnosis model; wherein the electrocardiogram diagnosis model is a trained neural network model; and
in case that the identification result indicates that the patient has sustained monomorphic ventricular tachycardia, determining that the patient meets the diagnosis condition for sustained monomorphic ventricular tachycardia.

According to the above method, optionally, the diagnosis rule library is established by a process comprising:
establishing an ontology model for sustained monomorphic ventricular tachycardia disease based on a preset biomedical and clinical medical knowledge library; determining etiology diagnosis knowledge point information based on the ontology model for sustained monomorphic ventricular tachycardia disease and a preset diagnosis and treatment standard knowledge information;
establishing a plurality of diagnosis rules based on the etiology diagnosis knowledge point information; and establishing the diagnosis rule library based on the plurality of diagnosis rules.

According to the above method, optionally, the diagnosis rule library is established by the process further comprising:
upon receiving a rule creation instruction sent by a permitted user, displaying a rule editing interface, to enable the permitted user to input a diagnosis rule through the rule editing interface; and upon receiving the diagnosis rule inputted by the permitted user, adding the diagnosis rule inputted by the permitted user to the diagnosis rule library.

According to the above method, optionally, the determining, based on the diagnosis information outputted by each target etiology diagnosis model, an etiology diagnosis result corresponding to the patient, comprises:
integrating respective predicted etiologies contained in respective diagnosis information to obtain a predicted etiology set;
ranking the respective predicted etiologies in the predicted etiology set according to a descending order of the predicted probabilities of the respective predicted etiologies in the predicted etiology set;
generating, based on the ranking of the respective predicted etiologies in the predicted etiology set, an etiology diagnosis result corresponding to the predicted etiology set; wherein the etiology diagnosis result corresponding to the predicted etiology set comprises each and every of the predicted etiologies in the predicted etiology set and the predicted probabilities thereof; and configuring the etiology diagnosis result corresponding to the predicted etiology set as the etiology diagnosis result corresponding to the patient.

According to the above method, optionally, the method further comprises:
in case that the target etiology diagnosis model in the model set comprises the first etiology diagnosis model, determining the etiology diagnosis rule corresponding to the diagnosis information outputted by the first etiology diagnosis model; and feeding back the etiology diagnosis rule to the user.

Provided is a device for diagnosing etiology, comprising:
a first determination unit, configured to determine electrocardiogram information corresponding to a patient in case that an etiology diagnosis for the patient is required; a judgment unit, configured to judge whether the patient meets a preset diagnosis condition for sustained monomorphic ventricular tachycardia based on the electrocardiogram information;
an information processing unit, configured to access case data of the patient in case that the patient meets the diagnosis condition for sustained monomorphic ventricular tachycardia, input the case data into an established etiology diagnosis information model, and obtain etiology diagnosis information corresponding to the patient upon processing by the etiology diagnosis information model;
a second determination unit, configured to determine a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method;
an etiology prediction unit, configured to input the etiology diagnosis information into the target etiology diagnosis model for each target etiology diagnosis model, and obtain diagnosis information outputted by the target etiology diagnosis model upon processing by the target etiology diagnosis model, wherein the diagnosis information comprises a plurality of predicted etiologies and predicted probabilities of respective predicted etiologies; and a third determination unit, configured to determine an etiology diagnosis result corresponding to the patient based on the diagnosis information outputted by each target etiology diagnosis model.

According to the above device, optionally, the judgment unit is particularly configured to:
determine a judgment mode selected by a user;
in case that the judgment mode is a preset manual judgment mode, determine whether the patient meets a preset symptom condition based on the electrocardiogram information;
in case that the patient meets the preset symptom condition, generate prompt information and access preset identification knowledge information; wherein the prompt information is configured to prompt that the patient has a symptom of sustained monomorphic ventricular tachycardia;
feed back the prompt information, the identification knowledge information and the electrocardiogram information to the user, and prompt the user to input a judgment result whether the patient has sustained monomorphic ventricular tachycardia; and in case that the judgment result inputted by the user indicates that the patient has sustained monomorphic ventricular tachycardia, determine that the patient meets the diagnosis condition for sustained monomorphic ventricular tachycardia.

According to the above device, optionally, the judgment unit is further configured to:
in case that the judgment mode is a preset intelligent judgment mode, access an electrocardiogram contained in the electrocardiogram information;
input the electrocardiogram into an established electrocardiogram diagnosis model, and obtain an identification result outputted by the electrocardiogram diagnosis model upon processing by the electrocardiogram diagnosis model; wherein the electrocardiogram diagnosis model is a trained neural network model; and in case that the identification result indicates that the patient has sustained monomorphic ventricular tachycardia, determine that the patient meets the diagnosis condition for sustained monomorphic ventricular tachycardia.

According to the above device, optionally, the diagnosis rule library is established by a process comprising:
establishing an ontology model for sustained monomorphic ventricular tachycardia disease based on a preset biomedical and clinical medical knowledge library; determining, based on the ontology model for sustained monomorphic ventricular tachycardia disease and a preset diagnosis and treatment standard knowledge information, etiology diagnosis knowledge point information;
establishing, based on the etiology diagnosis knowledge point information, a plurality of diagnosis rules; and establishing, based on the plurality of diagnosis rules, the diagnosis rule library.

According to the above device, optionally, the diagnosis rule library is established by the process further comprising:
upon receiving a rule creation instruction sent by a permitted user, displaying a rule editing interface, to enable the permitted user to input a diagnosis rule through the rule editing interface; and upon receiving the diagnosis rule inputted by the permitted user, adding the diagnosis rule inputted by the permitted user to the diagnosis rule library.

According to the above device, optionally, the third determination unit is particularly configured to:
integrate respective predicted etiologies contained in respective diagnosis information to obtain a predicted etiology set;
rank the respective predicted etiologies in the predicted etiology set according to a descending order of the predicted probabilities of the respective predicted etiologies in the predicted etiology set;
generate, based on the ranking of the respective predicted etiologies in the predicted etiology set, an etiology diagnosis result corresponding to the predicted etiology set; wherein the etiology diagnosis result corresponding to the predicted etiology set comprises each and every of the predicted etiologies in the predicted etiology set and the predicted probabilities thereof; and configure the etiology diagnosis result corresponding to the predicted etiology set as the etiology diagnosis result corresponding to the patient.

According to the above device, optionally, the third determination unit is further configured to:
in case that the target etiology diagnosis model in the model set comprises the first etiology diagnosis model, determine an etiology diagnosis rule corresponding to the diagnosis information outputted by the first etiology diagnosis model; and feed back the etiology diagnosis rule to the user.

Provided is a storage medium, comprising a stored instruction, wherein a device in which the storage medium is located is controlled to implement the above method for diagnosing etiology upon executing the instruction.

Provided is an electronic device, comprising a memory and one or more instructions, wherein the one or more instructions are stored on the memory and configured to be executed by one or more processors to implement the above method for diagnosing etiology.

A method for diagnosing etiology is provided according to the embodiments of the present disclosure, comprising: determining electrocardiogram information corresponding to a patient in case that an etiology diagnosis for the patient is required; determining, based on the electrocardiogram information, whether the patient meets a preset diagnosis condition for sustained monomorphic ventricular tachycardia; accessing case data of the patient in case that the patient meets the diagnosis condition for sustained monomorphic ventricular tachycardia, then inputting the case data into an established etiology diagnosis information model; obtaining etiology diagnosis information corresponding to the patient upon processing by the etiology diagnosis information model; determining a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method; inputting, for each target etiology diagnosis model, the etiology diagnosis information into the target etiology diagnosis model; obtaining, upon processing by the target etiology diagnosis model, diagnosis information outputted by the target etiology diagnosis model, wherein the diagnosis information comprises a plurality of predicted etiologies and predicted probabilities of the respective predicted etiologies; and determining, based on the diagnosis information outputted by each target etiology diagnosis model, an etiology diagnosis result corresponding to the patient. With the method provided by the embodiments of the present disclosure, it can be determined whether it is necessary to diagnose SMVT etiology for the patient based on the electrocardiogram information, and the etiology diagnosis result of SMVT can be determined based on case data of the patient and the pre-established etiology diagnosis model. It can assist clinicians in diagnosing the etiology of SMVT, thereby reducing the adverse effects of experience differences and workload on the etiology diagnosis and improving the accuracy of etiology diagnosis.

### BRIEF DESCRIPTION OF THE DRAWINGS

FIG. 1 is a method flowchart of a method for diagnosing etiology according to an embodiment of the present disclosure;

FIG. 2 is a method flowchart of a method for diagnosing etiology according to another embodiment of the present disclosure;

FIG. 3 is a schematic diagram of a process of diagnosing etiology according to an embodiment of the present disclosure;

FIG. 4 is a schematic diagram of a process of diagnosing etiology according to another embodiment of the present disclosure;

FIG. 5 is a schematic diagram of a structure of a device for diagnosing etiology according to an embodiment of the present disclosure; and

FIG. 6 is a schematic diagram of a structure of an electronic device according to an embodiment of the present disclosure.

### DETAILED DESCRIPTION OF EMBODIMENTS

The technical solutions in the embodiments of the present disclosure are clearly and thoroughly described below with reference to the accompanying drawings in the examples of the present disclosure. Apparently, the described examples are part of the embodiments of the present disclosure rather than all of them. Based on the embodiments in the present disclosure, all other embodiments obtained by those of ordinary skill in the art fall within the scope of protection of the present disclosure.

In the present disclosure, the term “comprise”, “include” or any other variation thereof is intended to cover a non-exclusive inclusion such that a process, method, product, or device comprising a set of elements comprises not only those elements, but also comprises other elements that are not explicitly listed, or also comprises elements inherent in such a process, method, product, or device. Without further limitations, an element defined by the phrase “comprising/including a/an . . . ” does not exclude the existence of additional identical elements in the process, method, product or device comprising the element.

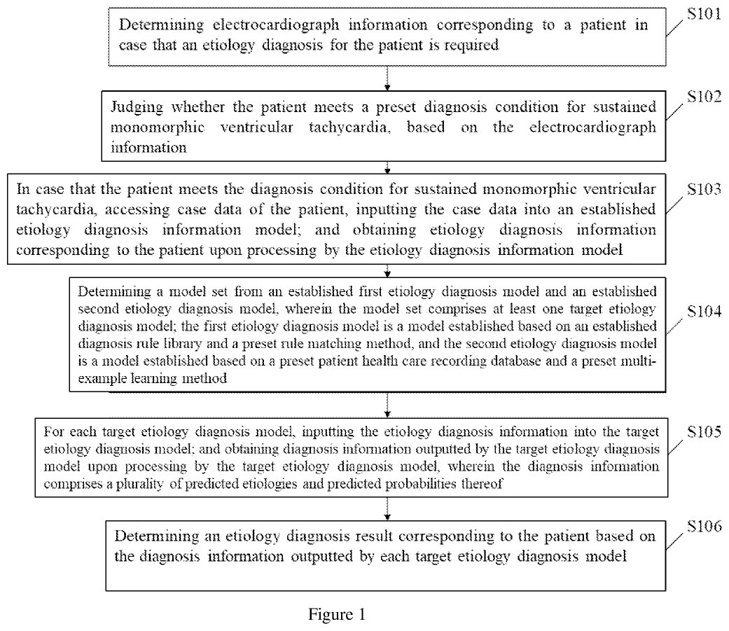

A device for diagnosing etiology is provided according to an embodiment of the present disclosure. The method may be applied to a system for supporting clinical decision, and an executor thereof may be a server of the system. The method flowchart of the method is shown in FIG. 1, which comprises steps S101 to S106 as follows.

In step S101, when it is required to diagnose the etiology for a patient, the electrocardiogram information corresponding to the patient is determined.

In the method provided by the embodiment of the present disclosure, the system for supporting clinical decision may be accessed to a hospital information system, wherein a user may select an identifier of the patient who needs to be diagnosed for etiology through the front end of the system, triggering the system for supporting clinical decision to diagnose etiology for the patient.

In the method provided by the embodiment of the present disclosure, when it is required to diagnose etiology for a patient, the electrocardiogram of the patient can be accessed from the database of the hospital information system, and the electrocardiogram report and other information data obtained by interpreting the electrocardiogram through a computer. The electrocardiogram of the patient, electrocardiogram report and other information data are used as the electrocardiogram information corresponding to the patient. Electrocardiogram (ECG) is a graph that records the electrical activity changes generated by each cardiac cycle of the heart from the body surfaces through an electrocardiogram machine, which is an existing technology, and will not be elaborated here.

In step S102, based on the electrocardiogram information, whether the patient meets a preset diagnosis condition for sustained monomorphic ventricular tachycardia is determined.

In the method provided by the embodiment of the present disclosure, a diagnosis condition for sustained monomorphic ventricular tachycardia may be set in advance, i.e., the condition used to determine whether the patient has SMVT and whether the etiology of SMVT needs to be diagnosed. Particularly, a model may be established to determine whether a patient has SMVT, and the identification result of the model is used to determine whether SMVT etiology diagnosis is needed. Alternatively, it may also prompt to manually determine whether the patient has SMVT based on the electrocardiogram information, and use the manual identification result to determine whether SMVT etiology diagnosis is required.

In step S103, in case that the patient meets the diagnosis condition for sustained monomorphic ventricular tachycardia, case data of the patient is accessed, and the case data is inputted into an established etiology diagnosis information model; and etiology diagnosis information corresponding to the patient is obtained upon the process by the etiology diagnosis information model.

In the method provided by the embodiment of the present disclosure, an etiology diagnosis information model is established in advance for extracting information data of specified attributes. On determining that the patient meets the diagnosis condition for sustained monomorphic ventricular tachycardia, case data of the patient can be read from the database, subsequently, case data of the patient can be inputted into the established etiology diagnosis information model, the established etiology diagnosis information model can perform information extraction on the case data of the patient, and information data outputted by the etiology diagnosis information model is the etiology diagnosis information corresponding to the patient. The etiology diagnosis information may include information data such as symptoms, past history, personal history, family history, physical signs, auxiliary examinations, laboratory tests, 12-lead information, and transthoracic ultrasound of the patient. The etiology diagnosis information model can directly extract structured information, and unstructured information can be extracted using natural language processing technology.

In step S104, a model set is determined from an established first etiology diagnosis model and an established second etiology diagnosis model. The model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method.

In the method provided by the embodiment of the present disclosure, the first etiology diagnosis model is established in advance based on a diagnosis rule library and a preset rule matching method associated with SMVT etiology diagnosis. Based on the inputted information, the first etiology diagnosis model may output corresponding SMVT etiology found through rule matching. Particularly, the first etiology diagnosis model may be established based on Bayesian network. In the method provided by the embodiment of the present disclosure, the second etiology diagnosis model is established in advance based on a preset patient health care recording database and a multi-example learning method. Based on the inputted information, the second etiology diagnosis model may output corresponding SMVT etiology found based on machine learning. In particular, case data of patient with SMVT in the patient health care recording database may be used as samples, and a model may be established and trained through a machine learning algorithm to obtain a second etiology diagnosis model.

In the method provided by the embodiment of the present disclosure, at least one etiology diagnosis model may be selected as the target etiology diagnosis model from the first etiology diagnosis model and the second etiology diagnosis model according to actual requirements to determine the model set. In particular, the user may select the etiological diagnosis model to be applied. It can be understood that the model set may only comprise one target etiology diagnosis model, and the target etiology diagnosis model is the first etiology diagnosis model or the second etiology diagnosis model. Alternatively, the model set may comprise two target etiology diagnosis models, i.e., the first etiology diagnosis model and the second etiology diagnosis model, respectively.

In step S105, for each target etiology diagnosis model, the etiology diagnosis information is inputted into the target etiology diagnosis model; and upon processing by the target etiology diagnosis model, diagnosis information outputted by the target etiology diagnosis model is obtained, wherein the diagnosis information comprises a plurality of predicted etiologies and predicted probabilities of respective predicted etiologies.

In the method provided by the embodiment of the present disclosure, the etiology diagnosis information corresponding to the patient is inputted into the target etiology diagnosis model. The target etiology diagnosis model may process the etiology diagnosis information and output corresponding diagnosis information, comprising multiple predicted etiologies and predicted probabilities thereof. It can be understood that in case that there is only one target etiology diagnosis model, the etiology diagnosis information is inputted into the model to obtain one piece of diagnosis information. In case that there are two target etiology diagnosis models, the etiology diagnosis information is inputted into each model respectively to obtain a total of two pieces of diagnosis information.

In step S106, based on the diagnosis information outputted by each target etiology diagnosis model, an etiology diagnosis result corresponding to the patient is determined.

In the method provided by the embodiment of the present disclosure, the etiology diagnosis result corresponding to the patient is determined based on the predicted etiology and the predicted probability corresponding to the predicted etiology contained in the diagnosis information. In particular, the predicted etiologies can be ranked in descending order of predicted probabilities, i.e., from predicted etiology with highest predicted probability to predicted etiology with lowest predicted probability, and the predicted etiologies ranked in order can be used as the corresponding etiology diagnosis result of the patient. The etiology diagnosis results can be fed back to the user through channels such as the system front end.

Based on the method provided by the embodiments of the present disclosure, when it is required to diagnose etiology for a patient, the electrocardiogram information corresponding to the patient is determined; based on the electrocardiogram information, whether the patient meets a preset diagnosis condition for sustained monomorphic ventricular tachycardia is determined; in case that the patient meets the diagnosis condition for sustained monomorphic ventricular tachycardia, case data of the patient is accessed and inputted into an established etiology diagnosis information model; then etiology diagnosis information corresponding to the patient is obtained upon processing by the etiology diagnosis information model; a model set is determined from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method; for each target etiology diagnosis model, the etiology diagnosis information is inputted into the target etiology diagnosis model; and upon processing by the target etiology diagnosis model, diagnosis information outputted by the target etiology diagnosis model is obtained, wherein the diagnosis information comprises a plurality of predicted etiologies and predicted probabilities of respective predicted etiologies; and based on the diagnosis information outputted by each target etiology diagnosis model, an etiology diagnosis result corresponding to the patient is determined. With the method provided by the embodiments of the present disclosure, it can be determined whether it is necessary to diagnose SMVT etiology for the patient based on the electrocardiogram information, and the etiology diagnosis result of SMVT can be determined based on case data of the patient and the pre-established etiology diagnosis model. It can assist clinicians in diagnosing the etiology of SMVT, thereby reducing the adverse effects of experience differences and workload on the etiology diagnosis and improving the accuracy of etiology diagnosis.

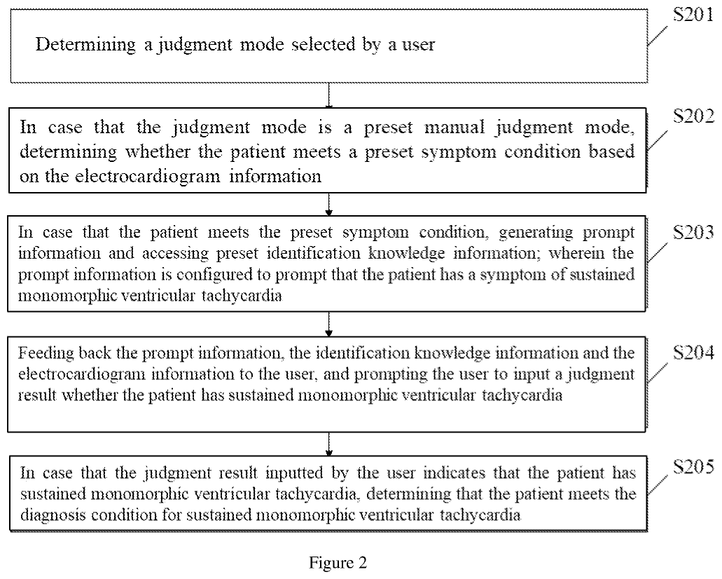

On the basis of the method shown in FIG. 1, a method for diagnosing etiology is provided according to another embodiment of the present disclosure. As shown in FIG. 2, in the method provided by the embodiment of the present disclosure, based on the electrocardiogram information mentioned in step S102, the process of determining whether the patient meets the preset diagnosis condition for sustained monomorphic ventricular tachycardia comprises steps S201 to S205 as follows.

In step S201, a judgment mode selected by a user is determined.

In the method provided by the embodiment of the present disclosure, the system may provide the user with optional judgment modes, including manual judgment mode, intelligent judgment mode, etc. The user may select the corresponding judgment mode through the system front end. The manual judgment mode refers to a mode in which the user manually examines the electrocardiogram information to identify whether the patient has SMVT. The intelligent judgment mode refers to a mode in which the system intelligently identifies whether the patient has SMVT through a preset algorithm.

In step S202, in case that the judgment mode is a preset manual judgment mode, whether the patient meets a preset symptom condition is determined based on the electrocardiogram information.

In the method provided by the embodiment of the present disclosure, a symptom condition can be set in advance according to the performance characteristics of SMVT in the electrocardiogram. In case that the judgment mode selected by the user is a preset manual judgment mode, the electrocardiogram report in the electrocardiogram information is read. Subsequently, it is determined whether the report content of the electrocardiogram report matches the condition content of the preset symptom condition, to determine whether the patient meets the preset symptom condition. For example, the QRS wave time and heart rate in the electrocardiogram may be used as a condition for determination, wherein the QRS wave time greater than 120 ms and the heart rate greater than 100 beats/min may be used as the symptom condition.

In step S203, in case that the patient meets the preset symptom condition, prompt information is generated, and preset identification knowledge information is accessed; wherein the prompt information is configured to prompt that the patient has symptom(s) of sustained monomorphic ventricular tachycardia.

In the method provided by the embodiment of the present disclosure, on determining that the patient meets the preset symptom condition, prompt information is generated to prompt the user that the patient may have SMVT. In addition, preset identification knowledge information can be accessed. The identification knowledge information is medical knowledge information used to identify whether a patient has SMVT.

In the method provided by the embodiment of the present disclosure, in case that the patient does not meet the preset symptom condition, the electrocardiogram information can be directly fed back to the user, and the user is prompted to make a determination.

In step S204, the prompt information, identification knowledge information and electrocardiogram information are fed back to the user, and the user is prompted to input a judgment result whether the patient has sustained monomorphic ventricular tachycardia.

In the method provided by the embodiment of the present disclosure, the prompt information, identification knowledge information, and electrocardiogram information of the patient can be fed back to the user through the system front end. Corresponding determination result controls are displayed, including a judgment result control used to indicate the patient has SMVT and a judgment result control used to indicate the patient does not have SMVT, to prompt the user to determine whether the patient has SMVT based on the above information and make a selection in the system.

In step S205, in case that the judgment result inputted by the user indicates that the patient has sustained monomorphic ventricular tachycardia, it is determined that the patient meets the diagnosis condition for sustained monomorphic ventricular tachycardia.

In the method provided by the embodiment of the present disclosure, in case that the judgment result inputted by the user indicates that the patient has SMVT, it is determined that the patient meets the diagnosis condition for sustained monomorphic ventricular tachycardia. In case that the judgment result inputted by the user indicates that the patient does not have SMVT, it is determined that the patient does not meet the diagnosis condition for sustained monomorphic ventricular tachycardia.

Based on the method provided by the above embodiments, the method provided by an embodiment of the present disclosure further comprises the following steps.

In case that the judgment mode is a preset intelligent judgment mode, an electrocardiogram contained in the electrocardiogram information is accessed.

In the method provided by the embodiment of the present disclosure, in case that the judgment mode selected by the user is a preset intelligent judgment mode, an electrocardiogram in the electrocardiogram information is read.

The electrocardiogram information is inputted into an established electrocardiogram diagnosis model, and an identification result outputted by the electrocardiogram diagnosis model is obtained upon processing by the electrocardiogram diagnosis model; wherein the electrocardiogram diagnosis model is a trained neural network model.

In the method provided by the embodiment of the present disclosure, an electrocardiogram diagnosis model is established in advance from the electrocardiogram sample data. The electrocardiogram diagnosis model can identify whether the inputted electrocardiogram has SMVT symptoms, to identify whether the patient has SMVT. In particular, the electrocardiogram diagnosis model is a trained neural network model, such as a convolutional neural networks (CNN) model or a recurrent neural network (RNN) model.

In the method provided by the embodiment of the present disclosure, the electrocardiogram of the patient is inputted into the electrocardiogram diagnosis model. After the electrocardiogram of the patient is identified and processed by the electrocardiogram diagnosis model, a corresponding identification result is outputted.

In case that the identification result indicates that the patient has sustained monomorphic ventricular tachycardia, it is determined that the patient meets the diagnosis condition for sustained monomorphic ventricular tachycardia.

In the method provided by the embodiment of the present disclosure, in case that the identification result outputted by the electrocardiogram diagnosis model indicates that the patient has SMVT, it is determined that the patient meets the diagnosis condition for sustained monomorphic ventricular tachycardia. In case that the identification result outputted by the electrocardiogram diagnosis model indicates that the patient does not have SMVT, it is determined that the patient does not meet the diagnosis condition for sustained monomorphic ventricular tachycardia.

Based on the method shown in FIG. 1, in the method provided by the embodiment of the present disclosure, the diagnosis rule library mentioned in step S104 is established by a process comprising the following steps:

Based on a preset biomedical and clinical medical knowledge library, an ontology model for sustained monomorphic ventricular tachycardia disease is established.

In the method provided by the embodiment of the present disclosure, based on the medical knowledge associated with SMVT in the biomedical and clinical medical knowledge library, logical rules between different knowledge are designed and encoded to form a computer-understandable knowledge system, thereby establishing an ontology model for sustained monomorphic ventricular tachycardia disease.

Based on the ontology model for sustained monomorphic ventricular tachycardia disease and preset diagnosis and treatment standard knowledge information, etiology diagnosis knowledge point information is determined.

In the method provided by the embodiment of the present disclosure, based on the ontology model for sustained monomorphic ventricular tachycardia disease, knowledge points of SMVT etiology diagnosis are identified from the preset diagnosis and treatment standard knowledge information. Then, a semantic relationship between the knowledge points and a logical determination are extracted to form etiology diagnosis knowledge point information of SMVT.

Based on the etiology diagnosis knowledge point information, a plurality of diagnosis rules are established.

In the method provided by the embodiment of the present disclosure, a multi-source, multi-granularity, and multi-dimensional diagnosis rule system is established based on the etiology diagnosis knowledge point information to obtain a plurality of diagnosis rules. The plurality of diagnosis rules may be set through logical rule operators such as AND, OR and NOT.

The diagnosis rule library is established based on the plurality of diagnosis rules.

In the method provided by the embodiment of the present disclosure, the diagnosis rule library is established based on the plurality of diagnosis rules.

Based on the method provided by the above embodiments, the method provided by an embodiment of the present disclosure further comprises the following steps:

Upon receiving a rule creation instruction sent by a permitted user, a rule editing interface is displayed, to enable the permitted user to input a diagnosis rule through the rule editing interface.

In the method provided by the embodiment of the present disclosure, a rule editing entrance is provided to support manual editing of diagnosis rules. Users who are permitted to edit diagnosis rules may send the rule creation instruction through the system front end. When receiving the rule creation instruction sent by the permitted user, the rule editing interface can be displayed in the front end, and a rule inputting box may be provided to enable the permitted user to input diagnosis rules through the interface.

Upon receiving the diagnosis rule inputted by the permitted user, the diagnosis rule inputted by the permitted user is added into the diagnosis rule library.

In the method provided by the embodiment of the present disclosure, after the permitted user inputs the diagnosis rule in the front end, the diagnosis rule can be submitted to the system. After the system receives the diagnosis rule inputted by the permitted user, the inputted diagnosis rule can be added to the diagnosis rule library for establishing the first etiology diagnosis model.

Based on the method provided by the embodiment of the present disclosure, it can provide clinicians with an entrance to construct diagnosis rules, enabling clinicians to participate in the editing of diagnosis rules, which is conducive to improving the matching between the diagnosis rule library and clinical diagnosis requirements, further improving the accuracy of diagnosis.

Based on the method shown in FIG. 1, in the method provided by the embodiment of the present disclosure, the process mentioned in step S106, i.e., determining an etiology diagnosis result corresponding to the patient based on the diagnosis information outputted by each target etiology diagnosis model, comprises the following steps:

Respective predicted etiologies contained in respective diagnosis information are integrated to obtain a predicted etiology set.

In the method provided by the embodiment of the present disclosure, in case that there are two target etiology diagnosis models, i.e., the first etiology diagnosis model and the second etiology diagnosis model are used for prediction simultaneously, it is necessary to integrate respective predicted etiologies in respective diagnosis information. In particular, repeated predicted etiologies may be merged, and the integrated respective predicted etiologies compose a predicted etiology set. For repeated predicted etiologies, the largest probability value among their corresponding predicted probabilities may be used as the predicted probability of the predicted etiology after merging.

In the method provided by the embodiment of the present disclosure, in case that only one target etiology diagnosis model is used for prediction, all predicted etiologies in the obtained diagnosis information may compose a predicted etiology set.

The respective predicted etiologies in the predicted etiology set are ranked according to a descending order of the predicted probabilities thereof in the predicted etiology set.

In the method provided by the embodiment of the present disclosure, the respective predicted etiologies in the predicted etiology set can be ranked according to a descending order of the predicted probabilities thereof in the predicted etiology set. The predicted etiology with a smaller predicted probability ranks after the predicted etiology with a larger predicted probability.

Based on the rank order of the respective predicted etiologies in the predicted etiology set, an etiology diagnosis result corresponding to the predicted etiology set is generated; the etiology diagnosis result corresponding to the predicted etiology set comprises each predicted etiology in the predicted etiology set and its predicted probability.

In the method provided by the embodiment of the present disclosure, the data of the predicted etiologies can be arranged according to the rank order of the predicted etiologies, and an etiology diagnosis result is generated. The etiology diagnosis result comprises each and every of the predicted etiologies ranked in the order and the predicted probabilities thereof.

The etiology diagnosis result corresponding to the predicted etiology set is used as the etiology diagnosis result corresponding to the patient.

In the method provided by the embodiment of the present disclosure, the etiology diagnosis result generated based on the respective predicted etiologies is used as the etiology diagnosis result corresponding to the current patient.

Based on the method shown in FIG. 1, the method provided by the embodiment of the present disclosure further comprises the following steps:

In case that the target etiology diagnosis model in the model set comprises the first etiology diagnosis model, the etiology diagnosis rule corresponding to the diagnosis information outputted by the first etiology diagnosis model is determined.

The the etiology diagnosis rule is fed back to the user.

In the method provided by the embodiment of the present disclosure, in case that the target etiology diagnosis model comprises the first etiology diagnosis model, i.e., the first etiology diagnosis model is currently used for etiology prediction, then the corresponding etiology diagnosis rule is determined based on the diagnosis information outputted by the first etiology diagnosis model. The etiology diagnosis rule can be fed back to the user while feeding back to the user the etiology diagnosis result corresponding to the patient. The etiology diagnosis rule refers to the diagnosis rule used to obtain diagnosis information by the first etiology diagnosis model during the process of predicting the etiology. In particular, the diagnosis rule may be the diagnosis rule in the diagnosis rule database.

Based on the method provided by the embodiment of the present disclosure, when outputting the etiology diagnosis result, the corresponding diagnosis rule can be outputted simultaneously, which can improve the interpretability of system diagnosis.

In order to better illustrate the method provided by the embodiment of the present disclosure, a method for diagnosing etiology is provided according to another embodiment of the present disclosure.

The method for diagnosing etiology provided by the embodiment of the present disclosure may be implemented based on a digital etiology diagnosis system (clinical decision support system). The etiology diagnosis system mainly implements system functions based on five modules.

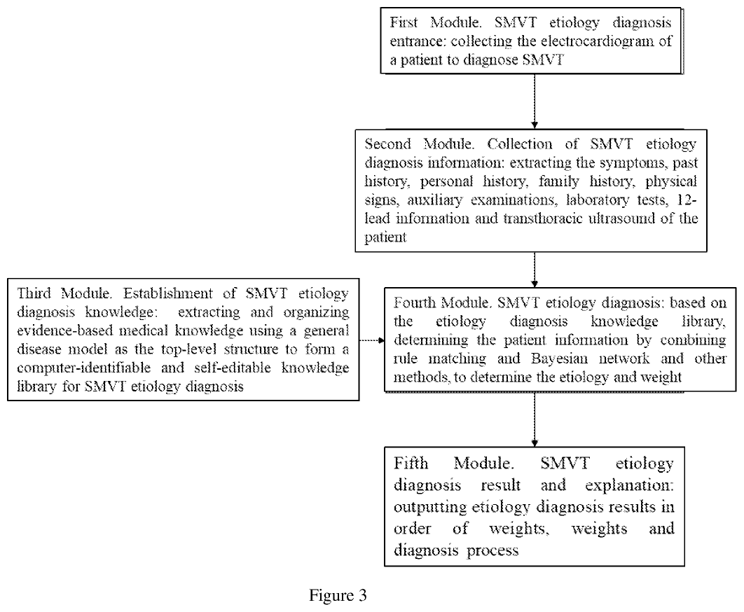

As shown in FIG. 3, illustrating with module functions, the etiology diagnosis process provided by the embodiment of the present disclosure mainly comprises the following steps:

### First Module: SMVT Etiology Diagnosis Entrance

The first module is used to determine a patient with SMVT and realize automatic connection between the digital etiology diagnosis system and the hospital information system. Without interfering with the normal work of clinicians, a patient with SMVT can be identified through electrocardiogram, which then triggers the SMVT etiology diagnosis system to assist clinicians in diagnosing the etiology of SMVT.

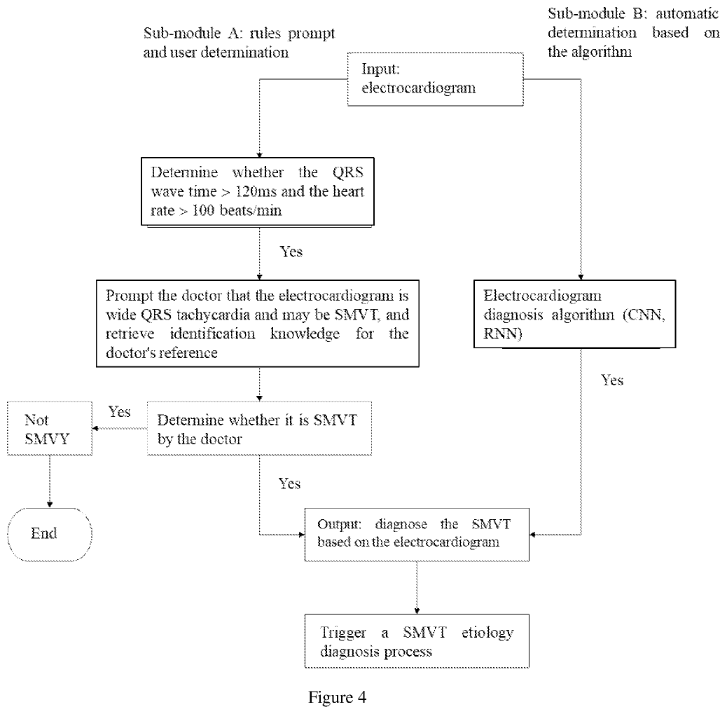

This module collects the electrocardiogram of a patient to diagnose SMVT. To identify a patient with SMVT based on electrocardiogram, there are mainly two optional sub-modules. One module is to provide a user (such as a clinician) with diagnosis rule information prompt, and the user evaluates the electrocardiogram (ECG) of the patient to determine whether it is SMVT. The other module is to determine whether the patient has SMVT using artificial intelligent algorithm (such as convolutional neural network and recurrent neural network) in combination with electrocardiogram (ECG). As shown in FIG. 4, the specific diagnosis process of the two sub-modules mainly comprises the following steps:

The electrocardiogram is inputted.

For sub-module A, a patient with SMVT is identified by using rule prompts and user decision. In particular, it is determined whether the QRS wave time in the electrocardiogram is greater than 120 ms, and whether the heart rate is greater than 100 beats/min. If so, the doctor is reminded that the electrocardiogram is wide QRS tachycardia, and the patient may have SMVT, and identification knowledge is retrieved for the doctor's reference. The doctor determines whether the patient has SMVT. If the doctor determines that it is not SMVT, the process ends. If the doctor determines that it is SMVT, the diagnosis result is outputted.

For sub-module B, a patient with SMVT is identified automatically based on the algorithm. In particular, the electrocardiogram is identified through the electrocardiogram diagnosis algorithm. If the identification result through algorithm is that the patient has SMVT, the diagnosis result is outputted.

In case that the outputted diagnosis result through the electrocardiogram is SMVT, the subsequent SMVT etiology diagnosis process is triggered.

### Second Module: Collection of SMVT Etiology Diagnosis Information

This module is used for collecting patient information. Medical knowledge and data of the patient in real world are associated with reference to the medical knowledge inputted by the SMVT etiology diagnosis information model (encompassing nine categories, i.e., the symptoms, past history, personal history, family history, physical signs, auxiliary examinations, laboratory tests, 12-lead information and transthoracic ultrasound of the patient). Using computer technology (etiology diagnosis information model), information such as symptoms, past history, personal history, family history, physical signs, auxiliary examinations, laboratory tests, 12-lead information and transthoracic ultrasound of the patient is extracted from the health care recordings of the patient with SMVT. Wherein, structured information may be extracted directly, and unstructured information may be extracted using natural language processing technology.

### Third Module: Construction of SMVT Etiology Diagnosis Knowledge

This module is a module that constructs pre-knowledge of the etiology diagnosis process, which extracts and organizes evidence-based medical knowledge using a general disease model as the top-level structure, to form a computer-identifiable and self-editable knowledge library for SMVT etiology diagnosis.

This module is used to construct the low-level knowledge library of the etiology diagnosis system, including a computer-understandable SMVT disease diagnosis knowledge library, SMVT-related medical knowledge recommendations (such as evidence-based medicine databases, clinical trials, patient case study reports, medical literature, etc.) and real-world SMVT patient datasets. Wherein, SMVT-related medical knowledge recommendations and patient datasets are mainly used to provide real-time information reference for doctors in the auxiliary diagnosis process. The former is obtained from third-party medical databases (such as BMJ Clinical Evidence, clinical trials (clinical trials database run by the U.S. National Library of Medicine and the U.S. Food and Drug Administration)), and PMC (PubMed Central) literature database). The latter is obtained from real patient case data from the hospital information system. The computer understandable SMVT disease diagnosis knowledge library is mainly used for the digitization of diagnostic knowledge and the construction of specific diagnosis rules, which is also the fundamental knowledge engine of the entire digital diagnosis system. The specific construction process comprises the following steps.

1. An ontology model for sustained monomorphic ventricular tachycardia disease is established, which contains relevant biomedical and clinical knowledge. The concepts of the ontology model may reflect different levels of information such as disease pathophysiological mechanisms and disease manifestations. The attributes of different concepts may be used to describe different states of the disease. Such knowledge is connected to existing biomedical knowledge systems (such as DO, HPO, MESH, ICD and UMLS) and encoded. Logical rules among different knowledge are configured and encoded to construct a computer-understandable knowledge system, which serves as a fundamental knowledge source of the entire digital diagnosis system.

2. Based on the fundamental knowledge described in step 1, knowledge points of SMVT etiology diagnosis are identified from unstructured medical knowledge in diagnosis and treatment specifications such as textbooks, clinical guidelines and expert experience, and the semantic relationships and logic determinations between the knowledge points are extracted, to form a multi-source, multi-granularity, and multi-dimensional diagnosis rule system. During this process, extraction of medical knowledge and establishment of diagnosis rules support both manual creation by users and automatic computer reasoning. For the manual creation process, the fundamental knowledge system established based on step 1 is computerized, and a diagnosis knowledge construction tool that supports manual configuration process and manual writing of rules (supporting logical rule operators such as AND, OR and NOT) is established. The diagnosis knowledge construction tool is interfaced with the diagnosis knowledge library in real time, which can provide clinicians with an entrance to the construction and review of disease diagnosis rules.

3. The etiology diagnosis model is established through two sub-modules.

A first sub-module: A computerized SMVT etiology diagnosis model is established based on the evidence-based medical knowledge obtained through steps 1 and 2, which encompasses nine categories information, i.e., the symptoms, past history, personal history, family history, physical signs, auxiliary examinations, laboratory tests, 12-lead information and transthoracic ultrasound of patients. The SMVT etiology diagnosis model can be automatically updated as the fundamental knowledge is updated and support users to customize, edit and review information. The SMVT etiology diagnosis model determines patient information through rule matching combined with weighting. Model construction may be achieved through methods such as a Bayesian network.

A second sub-module: An intelligent etiology diagnosis system is established based on the patient case database, and a patient etiology diagnosis model is established using a multi-example learning method.

### Fourth Module: SMVT Etiology Diagnosis

This module is used to diagnose the etiology of SMVT. Based on the etiology diagnosis knowledge library, patient information is determined by combining rule matching and Bayesian network and other methods, to determine the etiology and weight/predicted probability.

Application of the SMVT etiology diagnosis model: Correlation analysis is performed on the patient information extracted by the second module and the SMVT etiology diagnosis model established in the third module to determine the etiology of a patient with SMVT.

Application of the patient etiology diagnosis model: The patient information extracted by the second module 2 is inputted into the patient etiology diagnosis model established by the third module 3, and the etiology of a patient with SMVT is outputted.

### Fifth Module: Result and Explanation of SMVT Etiology Diagnosis

SMVT etiology diagnosis result is outputted in order of weights/predicted probabilities. For the etiology diagnosis obtained by applying the SMVT etiology diagnosis model in the fourth module, the corresponding diagnosis rules are outputted, i.e., the diagnosis process.

In the method provided by the embodiment of the present disclosure, in the part of construction of fundamental diagnosis knowledge, the fundamental knowledge of the SMVT disease ontology model is configured and computerized. Then diagnosis knowledge construction tools that are easy for users (clinicians) to use are embedded to optimize the digitization of fundamental knowledge and the processing of human-computer interaction, which improves the quality of fundamental knowledge and the flexibility of knowledge updating and editing.

An explanation module of the decision-making process is added to the output end to display the key process of etiological diagnosis in the form of a classification decision-tree, achieving real-time traceability and interpretability of decision-making results.

The etiology diagnosis entrance is configured as “one-click triggering” mode to interface the digital etiology diagnosis system with the hospital information system. Without interfering with the normal work of clinicians, the SMVT etiology diagnosis system is triggered through electrocardiogram or SMVT diagnosis conclusion to assist clinicians in diagnosing the etiology of SMVT.

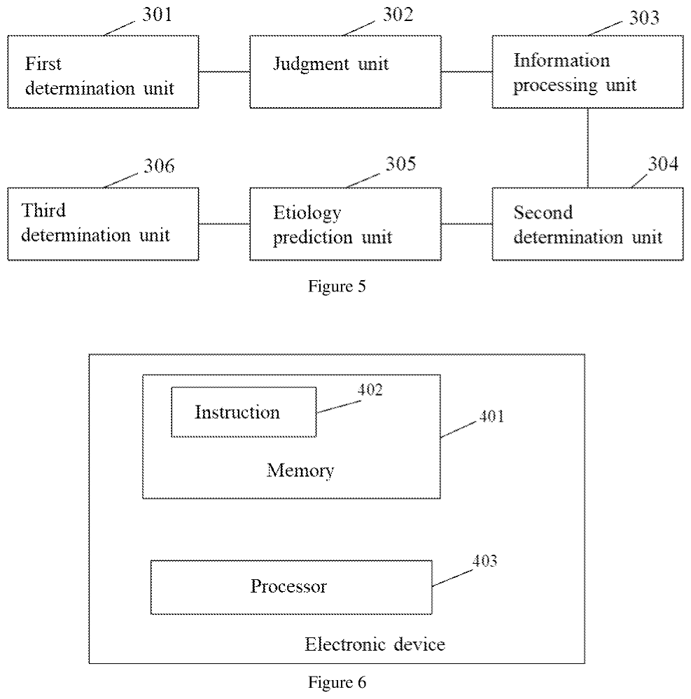

Corresponding to a method for diagnosing etiology shown in FIG. 1, a device for diagnosing etiology is further provided according to an embodiment of the present disclosure, which is used to implement the method shown in FIG. 1. A schematic diagram of the device structure is shown in FIG. 5, which comprises the following units:

A first determination unit 301, which is configured to determine electrocardiogram information corresponding to a patient in case that an etiology diagnosis for the patient is required.

A judgment unit 302, which is configured to determine whether the patient meets a preset diagnosis condition for sustained monomorphic ventricular tachycardia, based on the electrocardiogram information.

An information processing unit 303, which is configured to access case data of the patient in case that the patient meets the diagnosis condition for sustained monomorphic ventricular tachycardia, input the case data into an established etiology diagnosis information model, and obtain etiology diagnosis information corresponding to the patient upon the process by the etiology diagnosis information model.

A second determination unit 304, which is configured to determine a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method.

An etiology prediction unit 305, which is configured to input the etiology diagnosis information into the target etiology diagnosis model for each target etiology diagnosis model, and obtain diagnosis information outputted by the target etiology diagnosis model upon the process by the target etiology diagnosis model, wherein the diagnosis information comprises a plurality of predicted etiologies and predicted probabilities of respective predicted etiologies.

A third determination unit 306, which is configured to determine an etiology diagnosis result corresponding to the patient based on the diagnosis information outputted by each target etiology diagnosis model.

With the device provided by the embodiment of the present disclosure, it can determine whether it is necessary to diagnose SMVT etiology for the patient based on the electrocardiogram information. Based on case data of the patient and the pre-established etiology diagnosis model, the etiology diagnosis result of SMVT can be determined. It can assist clinicians in diagnosing the etiology of SMVT, thereby reducing the adverse effects of experience differences and workload on etiology diagnosis and improving the accuracy of etiology diagnosis.

Based on the device shown in FIG. 5, in the device provided by the embodiment of the present disclosure, the judgment unit is further configured to:
determine a judgment mode selected by a user;
in case that the judgment mode is a preset manual judgment mode, determine whether the patient meets a preset symptom condition based on the electrocardiogram information;
in case that the patient meets the preset symptom condition, generate prompt information and access preset identification knowledge information; wherein the prompt information is configured to prompt that the patient has a symptom of sustained monomorphic ventricular tachycardia;
feed back the prompt information, the identification knowledge information and the electrocardiogram information to the user, and prompt the user to input a judgment result whether the patient has sustained monomorphic ventricular tachycardia; and
in case that the judgment result inputted by the user indicates that the patient has sustained monomorphic ventricular tachycardia, determine that the patient meets the diagnosis condition for sustained monomorphic ventricular tachycardia.

Based on the device provided by the above embodiment, in the device provided by the embodiment of the present disclosure, the judgment unit is further configured to:
in case that the judgment mode is a preset intelligent judgment mode, access an electrocardiogram contained in the electrocardiogram information;
input the electrocardiogram into an established electrocardiogram diagnosis model, and obtain an identification result outputted by the electrocardiogram diagnosis model through the electrocardiogram diagnosis model; wherein the electrocardiogram diagnosis model is a trained neural network model; and
in case that the identification result indicates that the patient has sustained monomorphic ventricular tachycardia, determine that the patient meets the diagnosis condition for sustained monomorphic ventricular tachycardia.

Based on the device provided by the above embodiment, in the device provided by the embodiment of the present disclosure, the diagnosis rule library is established by a process comprising:
establishing an ontology model for sustained monomorphic ventricular tachycardia disease based on a preset biomedical and clinical medical knowledge library;
determining, based on the ontology model for sustained monomorphic ventricular tachycardia disease and a preset diagnosis and treatment standard knowledge information, etiology diagnosis knowledge point information;
establishing, based on the etiology diagnosis knowledge point information, a plurality of diagnosis rules; and
establishing, based on the plurality of diagnosis rules, the diagnosis rule library.

Based on the device provided by the above embodiment, in the device provided by the embodiment of the present disclosure, the diagnosis rule library is established by the process further comprising:
upon receiving a rule creation instruction sent by a permitted user, displaying a rule editing interface, to enable the permitted user to input a diagnosis rule through the rule editing interface; and
upon receiving the diagnosis rule inputted by the permitted user, adding the diagnosis rule inputted by the permitted user to the diagnosis rule library.

Based on the device provided by the above embodiment, in the device provided by the embodiment of the present disclosure, the third determination unit is particularly configured to:
integrate respective predicted etiologies contained in respective diagnosis information to obtain a predicted etiology set;
rank the respective predicted etiologies in the predicted etiology set according to a descending order of the predicted probabilities of the respective predicted etiologies in the predicted etiology set;
generate, based on the ranking of the respective predicted etiologies in the predicted etiology set, an etiology diagnosis result corresponding to the predicted etiology set; wherein the etiology diagnosis result corresponding to the predicted etiology set comprises each and every of the predicted etiologies in the predicted etiology set and the predicted probabilities thereof; and
configure the etiology diagnosis result corresponding to the predicted etiology set as the etiology diagnosis result corresponding to the patient.

Based on the device provided by the above embodiment, in the device provided by the embodiment of the present disclosure, the third determination unit is further configured to:
in case that the target etiology diagnosis model in the model set comprises the first etiology diagnosis model, determine an etiology diagnosis rule corresponding to the diagnosis information outputted by the first etiology diagnosis model;
feed back the etiology diagnosis rule to the user.

A storage medium is further provided according to an embodiment of the present disclosure. The storage medium comprises stored instructions. Upon executing the instructions, a device in which the storage medium is located is controlled to implement the above method for diagnosing etiology.

An electronic device is further provided according to an embodiment of the present disclosure. FIG. 6 illustrates a schematic diagram of the structure of the electronic device. In an embodiment, the electronic device comprises a memory 401, and one or more instructions 402, wherein one or more instructions 402 are stored on the memory 401 and are configured to be executed by one or more processors 403 to implement the following operations:
determining electrocardiogram information corresponding to a patient;
determining, based on the electrocardiogram information, whether the patient meets a preset diagnosis condition for sustained monomorphic ventricular tachycardia;
accessing, in case that the patient meets the diagnosis condition for sustained monomorphic ventricular tachycardia, case data of the patient, inputting the case data into an established etiology diagnosis information model, and obtaining, upon processing by the etiology diagnosis information model, etiology diagnosis information corresponding to the patient;
determining a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method;
inputting, for each target etiology diagnosis model, the etiology diagnosis information into the target etiology diagnosis model; and obtaining, through the target etiology diagnosis model, diagnosis information outputted by the target etiology diagnosis model, wherein the diagnosis information comprises a plurality of predicted etiologies and predicted probabilities of respective predicted etiologies; and
determining, based on the diagnosis information outputted by each target etiology diagnosis model, an etiology diagnosis result corresponding to the patient.

Each embodiment in this specification is described in a progressive manner, the same and similar parts of each embodiment can be referred to each other, and each embodiment focuses on the differences from other embodiments. In particular, for the devices or the device embodiments, since they are basically similar to the method embodiments, the description is relatively simple and the related parts can refer to the part of the description of the method embodiments. The devices and device embodiments described above are merely illustrative, wherein the units described as separate components may or may not be physically separated, and the components shown as units may or may not be physical units, that is, they can be located in one place, or they can be distributed to multiple network elements. Part or all of the modules can be selected according to practical needs to achieve the purpose of the solution of the embodiments. It can be understood and implemented by those skilled in the art without making inventive efforts.

It can be further understood by those skilled in the art that, the units and algorithm steps of the examples described in the embodiments disclosed herein can be implemented by electronic hardware, computer software or a combination thereof. In order to clearly illustrate the interchangeability of the hardware and software, the composition and steps of each example have been generally described according to their functions in the above descriptions. Whether the functions are performed by hardware or software depends on the specific applications and design constraints of the technical solution. Those skilled in the art may use different methods to realize the described functions for each specific application, but such realization should not be regarded as beyond the scope of the present disclosure.

The above are only specific embodiments of the present disclosure, and the protection scope of the present disclosure is not limited thereto. Modifications or substitutions can be easily considered by the skilled in the art within the technical scope disclosed by the present disclosure, which should be covered by the protection scope of the present disclosure. Hence, the protection scope of the present disclosure should be defined by the protection scope of the claims.

1. A device for diagnosing etiology, comprising:
a first determination unit, configured to determine electrocardiogram information corresponding to a patient in case that an etiology diagnosis for the patient is required;
a judgment unit, configured to judge whether the patient meets a preset diagnosis condition for sustained monomorphic ventricular tachycardia based on the electrocardiogram information;
an information processing unit, configured to access case data of the patient in case that the patient meets the diagnosis condition for sustained monomorphic ventricular tachycardia, input the case data into an established etiology diagnosis information model, and obtain etiology diagnosis information corresponding to the patient upon processing by the etiology diagnosis information model;
a second determination unit, configured to determine a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method;
an etiology prediction unit, configured to input the etiology diagnosis information into the target etiology diagnosis model for each target etiology diagnosis model, and obtain diagnosis information outputted by the target etiology diagnosis model upon processing by the target etiology diagnosis model, wherein the diagnosis information comprises a plurality of predicted etiologies and predicted probabilities of respective predicted etiologies; and
a third determination unit, configured to determine an etiology diagnosis result corresponding to the patient based on the diagnosis information outputted by each target etiology diagnosis model; and
a first display unit, configured to output and display the etiology diagnosis result.

2. The device according to claim 1, wherein the judgment unit is particularly configured to:
determine a judgment mode selected by a user;
in case that the judgment mode is a preset manual judgment mode, determine whether the patient meets a preset symptom condition based on the electrocardiogram information;
in case that the patient meets the preset symptom condition, generate prompt information and access preset identification knowledge information; wherein the prompt information is configured to prompt that the patient has a symptom of sustained monomorphic ventricular tachycardia;
feed back the prompt information, the identification knowledge information and the electrocardiogram information to the user, and prompt the user to input a judgment result whether the patient has sustained monomorphic ventricular tachycardia; and
in case that the judgment result inputted by the user indicates that the patient has sustained monomorphic ventricular tachycardia, determine that the patient meets the diagnosis condition for sustained monomorphic ventricular tachycardia.

3. The device according to claim 2, wherein the judgment unit is further configured to:
in case that the judgment mode is a preset intelligent judgment mode, access an electrocardiogram contained in the electrocardiogram information;
input the electrocardiogram into an established electrocardiogram diagnosis model, and obtain an identification result outputted by the electrocardiogram diagnosis model upon processing by the electrocardiogram diagnosis model; wherein the electrocardiogram diagnosis model is a trained neural network model; and
in case that the identification result indicates that the patient has sustained monomorphic ventricular tachycardia, determine that the patient meets the diagnosis condition for sustained monomorphic ventricular tachycardia.

4. The device according to claim 1, the device further comprises:
a first establishment unit, configured to establish an ontology model for sustained monomorphic ventricular tachycardia disease based on a preset biomedical and clinical medical knowledge library;
a fourth determination unit, configured to determine, based on the ontology model for sustained monomorphic ventricular tachycardia disease and a preset diagnosis and treatment standard knowledge information, etiology diagnosis knowledge point information;
a second establishment unit, configured to establish, based on the etiology diagnosis knowledge point information, a plurality of diagnosis rules; and
a third establishment unit, configured to establish, based on the plurality of diagnosis rules, the diagnosis rule library.

5. The device according to claim 4, wherein device further comprises:
a second display unit, configured to, upon receiving a rule creation instruction sent by a permitted user, display a rule editing interface, to enable the permitted user to input a diagnosis rule through the rule editing interface; and
an add unit, configured to, upon receiving the diagnosis rule inputted by the permitted user, add the diagnosis rule inputted by the permitted user to the diagnosis rule library.

6. The device according to claim 1, wherein the third determination unit is particularly configured to:
integrate respective predicted etiologies contained in respective diagnosis information to obtain a predicted etiology set;
rank the respective predicted etiologies in the predicted etiology set according to a descending order of the predicted probabilities of the respective predicted etiologies in the predicted etiology set;
generate, based on the ranking of the respective predicted etiologies in the predicted etiology set, an etiology diagnosis result corresponding to the predicted etiology set; wherein the etiology diagnosis result corresponding to the predicted etiology set comprises each and every of the predicted etiologies in the predicted etiology set and the predicted probabilities thereof; and
configure the etiology diagnosis result corresponding to the predicted etiology set as the etiology diagnosis result corresponding to the patient.

7. The device according to claim 1, wherein the third determination unit is further configured to:
in case that the target etiology diagnosis model in the model set comprises the first etiology diagnosis model, determine an etiology diagnosis rule corresponding to the diagnosis information outputted by the first etiology diagnosis model; and
feed back the etiology diagnosis rule to the user.

8. A non-transitory computer-readable storage medium, comprising a stored instruction, wherein, upon executing the instruction, a device in which the non-transitory computer-readable storage medium is located is controlled to implement the following steps:
determining electrocardiogram information corresponding to a patient;
determining, based on the electrocardiogram information, whether the patient meets a preset diagnosis condition for sustained monomorphic ventricular tachycardia;
accessing, in case that the patient meets the diagnosis condition for sustained monomorphic ventricular tachycardia, case data of the patient, then inputting the case data into an established etiology diagnosis information model; obtaining etiology diagnosis information corresponding to the patient upon processing by the etiology diagnosis information model;
determining a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method;
inputting, for each target etiology diagnosis model, the etiology diagnosis information into the target etiology diagnosis model; obtaining, upon processing by the target etiology diagnosis model, diagnosis information outputted by the target etiology diagnosis model, wherein the diagnosis information comprises a plurality of predicted etiologies and predicted probabilities of the respective predicted etiologies;
determining, based on the diagnosis information outputted by each target etiology diagnosis model, an etiology diagnosis result corresponding to the patient; and
outputting and displaying the etiology diagnosis result.

9. An electronic device, comprising a memory and one or more instructions, wherein the one or more instructions are stored on the memory and configured to be executed by one or more processors to implement the following steps:
determining electrocardiogram information corresponding to a patient;
determining, based on the electrocardiogram information, whether the patient meets a preset diagnosis condition for sustained monomorphic ventricular tachycardia;
accessing, in case that the patient meets the diagnosis condition for sustained monomorphic ventricular tachycardia, case data of the patient, then inputting the case data into an established etiology diagnosis information model; obtaining etiology diagnosis information corresponding to the patient upon processing by the etiology diagnosis information model;
determining a model set from an established first etiology diagnosis model and an established second etiology diagnosis model, wherein the model set comprises at least one target etiology diagnosis model; the first etiology diagnosis model is a model established based on an established diagnosis rule library and a preset rule matching method, and the second etiology diagnosis model is a model established based on a preset patient health care recording database and a preset multi-example learning method;
inputting, for each target etiology diagnosis model, the etiology diagnosis information into the target etiology diagnosis model; obtaining, upon processing by the target etiology diagnosis model, diagnosis information outputted by the target etiology diagnosis model, wherein the diagnosis information comprises a plurality of predicted etiologies and predicted probabilities of the respective predicted etiologies;
determining, based on the diagnosis information outputted by each target etiology diagnosis model, an etiology diagnosis result corresponding to the patient; and
outputting and displaying the etiology diagnosis result.
